(19) United States
(12) Patent Application Publication
(10) Pub. No.: US 2005/0008699 A1
(43) Pub. Date: Jan. 13, 2005

(54) Effervescent glucosamine composition

(76) Inventors: Fred Wehling, New Hope, MN (US); Mary Aldritt, Excelsior, MN (US)

Correspondence Address: Allison Johnson;Allison Johnson, P.A., 6016 Logan Ave. S., Minneapolis, MN, 55419, US

(21) Appl. No.: 10/618,413

(22) Filed: Jul. 11, 2003

(51) Int. Cl. A61K 31/737; A61K 31/525; A61K 31/51; A61K 9/46

(52) U.S. Cl. 424/466; 514/54; 514/62; 514/251; 514/276; 514/356; 514/474

(57) ABSTRACT

Disclosed is an effervescent composition that includes glucosamine, at least one component of an effervescent couple, and optionally chondroitin, and dissolves in excess water to form at least a uniform dispersion.

### BACKGROUND

The invention relates to dissolving glucosamine in an aqueous liquid.

A variety of treatments have been used in an attempt to relieve the aches and pains associated with joints and myofascial tissue (i.e., the skeletal muscles and their connective tissues) including osteoarthritis. Glucosamine and chondroitin are examples of two components that have been used to treat humans and animals suffering from such aches and pains. Glucosamine is an amino sugar extracted from the chitin in crab, lobster and shrimp shells.

Chondroitin is a biomacromolecule mucopolysaccharide extracted from the cartilage of various animals including sharks and bovine. Chondroitin tends to have a bad taste, which renders it unpalatable.

Compounds that are difficult to dissolve in water or unpalatable can be formulated as a solid dosage form such as a tablet or a capsule to help avoid unpleasant tastes and to address solubility problems. Tablets and capsules can be difficult to swallow for some people. Tablets and capsules preferably dissolve such that the active agent is released from the tablet or capsule such that it is available for use (e.g., absorption) by the body. As a result, use of the active agent by the body (e.g., through absorption) tends to be slower for active agents that are formulated into tablets and capsules relative to liquid dosage forms, and the active agent may not be completely used (e.g., absorbed) by the body prior to discharge.

Effervescent compositions are a useful dosage form for delivering active agents because they can be packaged in discreet and controlled quantities and tend to be easier to swallow relative to a tablet or capsule. Consumers tend to prefer effervescent compositions that dissolve in water. Compositions that coagulate can be aesthetically undesirable to the consumer and difficult to ingest.

It is difficult to incorporate an active agent in an effervescent composition while simultaneously achieving a composition that will exhibit a suitable dissolution profile, form an ingestible liquid composition, and exhibit an aesthetically acceptable appearance to the consumer. It is also difficult to formulate an effervescent composition that further is capable of being formed into a tablet that maintains its integrity until use.

### SUMMARY

In one aspect the invention features an effervescent composition that includes glucosamine, chondroitin, and an effervescent agent. In one embodiment, the composition dissolves in water to form a clear solution. In another embodiment, about 7 grams (g) of the composition dissolves in from 200 mL to 400 mL water to form a clear solution. In other embodiments, the composition dissolves in water to form a substantially uniform dispersion.

In some embodiments, the composition further includes calcium (e.g., calcium lactate). In other embodiments, the composition further includes at least 5% by weight calcium lactate and no greater than 10% by weight calcium carbonate.

In one embodiment, the composition further includes magnesium. In other embodiments, the composition further includes vitamin. In some embodiments, the vitamin is selected from the group consisting of vitamin C, thiamin, riboflavin, nicotinic acid, pantothenic acid, pyridoxine, biotin, folic acid, niacin, vitamin B12, lipoic acid, ascorbic acid, vitamin A, vitamin D, vitamin E, and vitamin K, and combinations thereof.

In other embodiments, the composition includes at least 2% by weight glucosamine. In some embodiments, the composition includes at least 1% by weight chondroitin.

In one embodiment the composition includes at least 2% by weight glucosamine and at least 1% by weight chondroitin. In other embodiments, the composition includes from 3% by weight to 25% by weight glucosamine and from 2% by weight to 10% by weight chondroitin.

In another aspect, the invention features an effervescent composition that includes glucosamine, chondroitin, calcium lactate, magnesium sulfate, and an effervescent agent. The composition dissolves in water to form a solution. In one embodiment, the composition disperses in water to form a substantially uniform dispersion.

In another aspect, the invention features an effervescent composition that includes glucosamine and an effervescent agent, the composition, when placed in excess water, dissolves to form at least a substantially uniform dispersion. In one embodiment, the composition dissolves to form a solution.

In one embodiment the effervescent composition is in the form of a tablet. In some embodiments, the tablet has a hardness of at least 6 kilopounds. In other embodiments, the tablet has a hardness of from about 6 kilopounds to about 10 kilopounds.

In another embodiment, the effervescent composition is in the form of a powder.

The invention features an effervescent glucosamine composition that dissolves in excess water and forms a substantially uniform dispersion as viewed by the naked eye. The invention also features a composition that provides a single dosage form for the simultaneous administration of multiple active agents including glucosamine and chondroitin, and optionally calcium, magnesium, vitamins, and combinations thereof. The effervescent composition exhibits a good dissolution or dispersion rate and forms a palatable aqueous liquid composition.

Other features and advantages will be apparent from the following description of the preferred embodiments and from the claims.

### Glossary

In reference to the invention, these terms have the meanings set forth below:

The term “effervescent composition” refers to a composition that gives off a gas (e.g., carbon dioxide) when placed in an aqueous liquid.

### DETAILED DESCRIPTION

The effervescent composition includes glucosamine, an effervescent agent, and optionally chondroitin, calcium lactate, calcium carbonate, magnesium sulfate, and combinations thereof. The effervescent composition is water soluble such that it dissolves (i.e., dissolves, disperses, disintegrates or a combination thereof) in water. Preferably the effervescent composition forms at least a substantially uniform dispersion, more preferably a clear solution, when dissolved in a sufficient amount of water. The uniformity and clarity of the composition is determined by viewing with the naked eye. Preferably the effervescent composition dissolves in excess water at room temperature (about 22° C.) in less than two minutes. The composition preferably is self-mixing, i.e., when excess water is added to the effervescent composition, the effervescent composition will dissolve on its own without mixing or stirring from another source. The composition is palatable and can be easily swallowed. The effervescent composition preferably provides a liquid composition having a pH from 3 to 5 when dissolved in water.

Suitable forms of glucosamine include, e.g., glucosamine, glucosamine salts, and mixtures thereof. Suitable salts of the glucosamine include the hydrochloride, sulfate, nitrate and iodide. Preferred forms of glucosamine include glucosamine sulfate, glucosamine hydrochloride, N-acetylglucosamine, and the potassium chloride and sodium chloride salts thereof. Glucosamine can be obtained from various sources including, e.g., shell fish sources and fermentation processes. Preferably the composition includes at least about 2% by weight, from about 3% by weight to about 25% by weight, or even from about 5% by weight to about 15% by weight glucosamine.

Chondroitin is available in a variety of forms including, e.g., chondroitin, chondroitin salts, and mixtures thereof. Preferably chondroitin is the form of chondroitin sulfate. Useful examples of chondroitin sulfate include Type A (chondroitin-4-sulfate), Type B (chondroitin-5-sulfate), Type C (chondroitin-6-sulfate), and combinations thereof. Chondroitin can be obtained through fermentation or extraction from bovine trachea, other bovine, porcine, and shark sources. The present inventors have discovered that chondroitin obtained from a bovine source provides a more palatable composition relative to chondroitin obtained from shark and porcine sources. Preferably the composition includes at least about 0.5% by weight, from about 1% by weight to about 15% by weight, or even from about 2% by weight to about 10% by weight chondroitin.

The composition preferably includes calcium lactate in an amount of at least about 5% by weight, from about 10% by weight to about 45% by weight, or even from about 20% by weight to about 40% by weight.

The composition preferably includes calcium carbonate in an amount of at least about 2% by weight, from about 3% by weight to about 15% by weight, or even from about 4% by weight to about 10% by weight.

The amount of calcium including calcium lactate and/or calcium carbonate present in the composition is preferably sufficient to provide at least 50% of the recommended daily allowance of calcium.

The magnesium can be in a variety of forms including, e.g., magnesium sulfate, magnesium carbonate, magnesium oxide, magnesium citrate, magnesium lactate, and magnesium amino acid chelate. Magnesium is preferably present in the form of magnesium sulfate. The amount of magnesium sulfate in the composition preferably is at least about 5% by weight, from about 7% by weight to about 30% by weight, or even from about 10% by weight to about 25% by weight. The amount of magnesium in the composition is preferably sufficient to provide at least 50% of the recommended daily allowance of magnesium.

The effervescent agent preferably is at least one component of an effervescent couple that includes an acid and a base. The effervescent couple is activated when contacted with water, e.g., when the powder or tablet is placed in a glass of water. The water liberates the acid and base and enables the acid and base to react with each other to produce carbon dioxide gas, which imparts carbonation to the aqueous composition. Examples of useful acids include citric acid, ascorbic acid, aspartic acid, malic acid, adipic acid, tartaric acid, fumaric acid, succinic acid, sodium acid pyrophosophate, lactic acid, hexamic acid, amino acids, and acid salts and acid anhydrides thereof, and mixtures thereof. Examples of useful acid anhydrides include citraconic anhydride, glucono-D-lactone, and succinic anhydride. Examples of useful acid salts include potassium bitartrate, acid citrate salts, sodium dihydrogen phosphate, disodium dihydrogen phosphate, sodium acid sulfite, and combinations thereof. Preferably acid is present in the composition in an amount of from 10% by weight to about 60% by weight, from about 15% by weight to about 50% by weight, or even from about 25% by weight to about 40% by weight.

The base preferably is capable of generating carbon dioxide. Examples of suitable carbonate bases include sodium bicarbonate, sodium carbonate, sodium sesquicarbonate, potassium carbonate, potassium bicarbonate, calcium carbonate, magnesium carbonate, magnesium oxide, sodium glycine carbonate, L-lysine carbonate, arginine carbonate, zinc carbonate, zinc oxide, amino acid carbonates, and mixtures thereof. The composition preferably includes base in an amount of from 10% by weight to about 60% by weight, from about 15% by weight to about 50% by weight, or even from about 25% by weight to about 40% by weight.

The effervescent composition can optionally include a variety of additional active agents including, e.g., vitamins, amino acids, pharmaceutical agents, minerals, dietary supplements, and combinations thereof. Suitable vitamins include, e.g., ascorbic acid (vitamin C), aspartic acid, thiamin, riboflavin, nicotinic acid, pantothenic acid, pyridoxine, biotin, folic acid, niacin, vitamin B12, lipoic acid, vitamin A, vitamin D, vitamin E and vitamin K and coenzymes thereof, choline, camitine, and alpha, beta, and gamma carotenes. Examples of coenzymes include thiamine pyrophosphates, flavin mononucleotide, flavin adenine dinucleotide, nicotinamide adenine dinucleotide, nicotinamide adenine dinucleotide phosphate coenzyme A pyridoxal phosphate, biocytin, tetrahydrofolic acid, coenzyme B12, lipoyllysine, 11-cis-retinal, and 1,25-dihydroxycholecalciferol and mixtures.

Suitable amino acids include, e.g., L-tyrosine, isoleucine, ornithine, glutamine, phenylalanine, leucine, lysine, methionine, threonine, taurine, tryptophan, valine, alanine, glycine, arginine, histidine, cysteine, asparagine, proline and serine, and mixtures thereof.

Examples of minerals include iron, zinc, selenium, copper, iodine, phosphorus, chromium and mixtures thereof.

Suitable dietary supplements include, e.g., bee pollen, bran, wheat germ, kelp, cod liver oil, ginseng, and fish oils, amino-acids, proteins, vitamins, minerals alpha-glycerylphosphorylcholine, acetyl-L-carnitine and salts thereof, docosahexaenoic acid, glucosamine, chondroitin, methylsulfonylmethane, and mixtures thereof.

The composition can also include other ingredients including, e.g., flavor agents, fillers, surfactants (e.g., polysorbate 80 and sodium lauryl sulfate), color agents including, e.g., dyes and pigments, and sweeteners.

Useful flavor agents include natural and synthetic flavoring sources including, e.g., volatile oils, synthetic flavor oils, flavoring aromatics, oils, liquids, oleoresins and extracts derived from plants, leaves, flowers, fruits, stems and combinations thereof. Useful flavor agents include, e.g., citric oils, e.g., lemon, orange, grape, lime and grapefruit, fruit essences including, e.g., apple, pear, peach, grape, strawberry, raspberry, cherry, plum, pineapple, apricot, and other fruit flavors. Other useful flavor agents include, e.g., aldehydes and esters (e.g., benzaldehyde (cherry, almond)), citral, i.e., alpha-citral (lemon, lime), neral, i.e., beta-citral (lemon, lime), decanal (orange, lemon), aldehyde C-8 (citrus fruits), aldehyde C-9 (citrus fruits), aldehyde C-12 (citrus fruits), tolyl aldehyde (cherry, almond), 2,6-dimethyloctanal (green fruit), 2-dodedenal (citrus, mandarin) and mixtures thereof.

Useful color agents include, e.g., food, drug and cosmetic (FD&C) colors including, e.g., dyes, lakes, and certain natural and derived colorants. Useful lakes include dyes absorbed on aluminum hydroxide and other suitable carriers.

Useful sweetening agents include stevia, sugars such as sucrose, glucose, invert sugar, fructose, ribose, tagalose, sucralose, malitol, erythritol, xylitol, and mixtures thereof, saccharin and its various salts (e.g., sodium and calcium salt of saccharin), cyclamic acid and its various salts, dipeptide sweeteners (e.g., aspartame), acesulfame potassium, dihydrochalcone, glycyrrhizin, and sugar alcohols including, e.g., sorbitol, sorbitol syrup, mannitol and xylitol, and combinations thereof.

The effervescent composition can be in a variety of forms including, e.g., powder (e.g., a free flowing granulation), tablet, capsule, pellet and composite. Preferred effervescent tablets have a hardness of at least 3 kilopounds (Kp), preferably at least 6 Kp, from about 6 Kp to about 10 Kp, or even from about 6 Kp to about 8 Kp, as measured on a standard hardness tester fitted with a strain gauge.

When in the form of a tablet, the composition preferably includes binder, lubricant, and combinations thereof. Examples of suitable binders include, e.g., starches, natural gums, cellulose gums, microcrystalline cellulose, methylcellulose, cellulose ethers, sodium carboxymethylcellulose, ethylcellulose, gelatin, dextrose, lactose, sucrose, sorbitol, mannitol, polyethylene glycol, polyvinylpyrrolidone, pectins, alginates, polyacrylamides, polyvinyloxoazolidone, polyvinylalcohols and mixtures thereof.

Where present, the composition includes a sufficient amount of binder to assist in holding the components of the composition together in the form of a tablet. When present, the composition preferably includes binder in an amount of from 10% by weight to about 60% by weight, from about 15% by weight to about 50% by weight, or even from about 25% by weight to about 40% by weight.

Various lubricants are suitable for use in the composition including water dispersible, water soluble, water insoluble lubricants and combinations thereof. Preferred lubricants are water soluble. Examples of useful water soluble lubricants include sodium benzoate, polyethylene glycol, L-leucine, adipic acid, and combinations thereof. The composition can also include water insoluble lubricants including, e.g., stearates (e.g., magnesium stearate, calcium stearate and zinc stearate), oils (e.g., mineral oil, hydrogenated and partially hydrogenated vegetable oils, and cotton seed oil) and combinations thereof. Other water insoluble lubricants include, e.g., animal fats, polyoxyethylene monostearate, talc, and combinations thereof.

The composition preferably includes a sufficient amount of lubricant to enable the composition to be formed into tablets and released from a high speed tableting press in the form of a tablet. When present, the composition preferably includes lubricant in an amount of from 1% by weight to about 15% by weight, from about 1% by weight to about 12% by weight, from about 2% by weight to about 10% by weight, or even from about 3% by weight to about 8% by weight.

The effervescent composition is preferably stored in a moisture-proof package including, e.g., sealed metal foil pouches, blister packs, and desiccant capped tubes. The composition can be administered by dissolving the composition in excess water, e.g., an eight ounce glass of tap water, to form an aqueous solution and ingested. After addition of the effervescent composition to an aqueous liquid, the composition optionally can be stirred to facilitate dispersion and/or dissolution in the aqueous liquid.

The invention will now be described by way of the following examples.

### EXAMPLES

### Example 1

An effervescent composition in the form of a powder was prepared by combining 2290 mg calcium lactate (13.1% calcium), 1400 mg citric acid, 1389 mg magnesium sulfate (14.4% magnesium), 757 mg glucosamine hydrochloride, 315 mg powdered sodium bicarbonate (27% sodium), 520 mg calcium carbonate (38.5% calcium), 241 mg chondroitin sulfate (83%), 100 mg sodium carbonate grade 50 (43% sodium), 125 mg orange flavoring, 60 mg beet root powder, 60 mg stevia, 25 mg grapefruit flavoring, 20 mg boron amino acid chelate (5% boron), 2.4 mg dry vitamin D3, and 2.2 mg riboflavin 5-phosphate (67.5% riboflavin) in a blender with mixing.

The 7306.6 mg of the effervescent powder was placed in the bottom of a glass vessel and 200 ml of water was added. The powder was observed to effervesce and dissolve in less than two minutes.

### Example 2

An effervescent composition in the form of a tablet is prepared by combining 1145 mg calcium lactate (13.1% calcium), 1000 mg citric acid, 800 mg sorbitol instant, 694.5 mg magnesium sulfate (14.4% magnesium), 375 mg glucosamine hydrochloride, 315 mg powdered sodium bicarbonate (27% sodium), 260 mg calcium carbonate (38.5% calcium), 200 mg polyethylene glycol, 111 mg chondroitin sulfate (83%), 100 mg sodium benzoate, 100 mg sodium carbonate grade 50 (43% sodium), 100 mg orange flavoring, 60 mg beet root powder, 60 mg stevia, 50 mg tangerine flavoring, 10 mg boron amino acid chelate (5%), 10 mg acesulfame potassium, 1.2 mg dry vitamin D3, and 2.2 mg riboflavin 5-phosphate (67.5% riboflavin) in a blender with mixing.

The formulation is mixed for 20 minutes and then transferred to a tablet press having a one inch tool to form tablets weighing from approximately 3.5 g to 5.5 g. The tablets are pressed to a hardness of at least four kilopounds.

A tablet is then placed in excess water, approximately 200 ml. The tablet is expected to dissolve in less than two minutes.

Other embodiments are within the claims.

1. An effervescent composition comprising:
glucosamine;
chondroitin; and
an effervescent agent.

2. The effervescent composition of claim 1, wherein said composition dissolves in water to form a clear solution.

3. The effervescent composition of claim 1, wherein 7 g of said composition dissolves in from 200 mL to 400 mL water to form a clear solution.

4. The effervescent composition of claim 1, wherein said composition dissolves in water to form a substantially uniform dispersion.

5. The effervescent composition of claim 1, further comprising calcium.

6. The effervescent composition of claim 1, further comprising calcium lactate.

7. The effervescent composition of claim 1, further comprising at least 5% by weight calcium lactate and no greater than 10% by weight calcium carbonate.

8. The effervescent composition of claim 1, further comprising magnesium.

9. The effervescent composition of claim 4, further comprising magnesium.

10. The effervescent composition of claim 1, further comprising vitamin.

11. The effervescent composition of claim 10, wherein said vitamin is selected from the group consisting of vitamin C, thiamin, riboflavin, nicotinic acid, pantothenic acid, pyridoxine, biotin, folic acid, niacin, vitamin B12, lipoic acid, ascorbic acid, vitamin A, vitamin D, vitamin E, and vitamin K, and combinations thereof.

12. The effervescent composition of claim 1, comprising at least 2% by weight glucosamine.

13. The effervescent composition of claim 1, comprising at least 1% by weight chondroitin.

14. The effervescent composition of claim 1, comprising
at least 2% by weight glucosamine and
at least 1% by weight chondroitin.

15. The effervescent composition of claim 1, comprising
from 3% by weight to 25% by weight glucosamine and
from 2% by weight to 10% by weight chondroitin.

16. An effervescent composition comprising:
glucosamine;
chondroitin;
calcium lactate;
magnesium sulfate; and
an effervescent agent.

17. The effervescent composition of claim 16, wherein said composition dissolves in water to form a solution.

18. The effervescent composition of claim 16, wherein said composition disperses in water to form a substantially uniform dispersion.

19. An effervescent composition comprising:
glucosamine; and
an effervescent agent,
said composition, when placed in excess water, dissolves to form at least a substantially uniform dispersion.

20. The effervescent composition of claim 19, wherein said composition dissolves to form a solution.

21. A tablet comprising the effervescent composition of claim 1.

22. The tablet of claim 21, wherein the tablet has a hardness of at least 6 kilopounds.

23. The tablet of claim 21, wherein the tablet has a hardness of from about 6 kilopounds to about 10 kilopounds.

24. A powder comprising the effervescent composition of claim 1.
